(19) United States
(12) Patent Application Publication
(10) Pub. No.: US 2014/0205583 A1
(43) Pub. Date: Jul. 24, 2014

(54) USE OF PLP WITH PEG-rMETase IN VIVO FOR ENHANCED EFFICACY

(71) Applicant: AntiCancer, Inc., San Diego, CA (US)

(72) Inventors: Shigeo YAGI, San Diego, CA (US); Zhijian YANG, San Diego, CA (US); Shukuan LI, San Diego, CA (US); Xinghua SUN, San Diego, CA (US); Yuying TAN, San Diego, CA (US)

(73) Assignee: ANTICANCER, INC., San Diego, CA (US)

(21) Appl. No.: 14/162,655

(22) Filed: Jan. 23, 2014

Related U.S. Application Data
(63) Continuation of application No. 12/824,116, filed on Jun. 25, 2010, now abandoned.
Application No. 12/824,116 is a continuation of application No. 10/891,662, filed on Jul. 15, 2004, now abandoned.
(60) Provisional application No. 60/491,792, filed on Jul. 31, 2003.

(51) Int. Cl. A61K 38/51; A61K 31/675

(52) U.S. Cl. CPC A61K 38/51; A61K 31/675
USPC 424/94.3

(57) ABSTRACT

This invention relates to methods of modifying pyridoxal 5′ phosphate (PLP) dependent enzymes to extend the serum half-life of the enzyme, extend the in vivo period of methionine depletion in a host, and decrease the immunogenicity of the enzyme. A preferred PLP-dependent enzyme to be modified is a methioninase, preferably a recombinant methioninase (rMETase). The invention further relates to compositions comprising a modified PLP-dependent enzyme and methods of using the same.

### CROSS-REFERENCE TO RELATED APPLICATION

This application is a continuation of U.S. patent application Ser. No. 12/824,116 filed Jun. 25, 2010, currently pending, which is a continuation of U.S. patent application Ser. No. 10/891,662 filed on Jul. 15, 2004, now abandoned, which claims the benefit of priority to U.S. Provisional Patent Application No. 60/491,762, filed Jul. 31, 2003, each application is hereby incorporated by reference in its entirety.

### STATEMENT OF RIGHTS TO INVENTIONS MADE UNDER FEDERALLY SPONSORED RESEARCH

This study was supported in part by National Cancer Institute grant number 1 R43 CA86166-01.

### TECHNICAL FIELD

This invention relates to methods of modifying pyridoxal 5′ phosphate (PLP) dependent enzymes to extend the serum half-life of the enzyme, extend the in vivo period of methionine depletion in a host, and decrease the immunogenicity of the enzyme. A preferred PLP-dependent enzyme to be modified is a methioninase, preferably a recombinant methioninase (rMETase). The invention further relates to compositions comprising a modified PLP-dependent enzyme and methods of using the same.

### BACKGROUND ART

Previous studies have extensively documented that a broad range of human tumors are sensitive to rMETase in vitro. The IC₅₀ for rMETase was several-fold less for a wide variety of cancer cell lines compared to non-neoplastic cells. The rMETase-induced sensitivity was particularly exquisite for breast, kidney, colon, lung and prostate cell lines (Tan, Y., et al., Protein Expr. Purif. (1997) 9:233-245, Tan, Y., et al., Clin. Cancer Res. (1999) 5:2157-2163). Subsequent evaluation of a variety of tumor cell lines in mouse xenograph models demonstrated similar sensitivity to rMETase. In addition, rMETase-mediated plasma methionine depletion resulted in an increase in tumor sensitivity to several different chemotherapeutic agents. Unfortunately, the immunogenicity and short serum half-life of rMETase has limited the development of this important therapeutic agent.

### DISCLOSURE OF THE INVENTION

The description below relates to methods of modifying PLP-dependent enzymes such as rMETase, to extend the serum half-life of the enzyme, extend the in vivo period of methionine depletion in a host, and decrease the immunogenicity of the enzyme. The invention further relates to compositions comprising the modified PLP-dependent enzymes and methods of using the same.

Methoxypolyethylene glycol succinimidyl glutarate PEG (MEGC-PEG) was used to impart new molecular and functional advantages to rMETase. A remarkable prolongation of effective enzyme serum half-life and duration of methionine depletion was achieved by PEGylation of rMETase. The immunogenicity of the enzyme was also advantageously reduced as a result of PEGylation. It was also noted that PEGylation of rMETase decreased enzyme dependency on pyridoxal 5′ phosphate (PLP), a co-factor required for rMETase function.

### BRIEF DESCRIPTION OF THE DRAWINGS

FIG. 1 shows the chemical structure of MEGC-50HS-PEG (methoxypolyethylene glycol succinimidyl glutarate-PEG).

FIG. 2 shows a SDS-PAGE of naked and PEGylated rMETase.

FIGS. 3A-D show several plots of MALDI spectrum of naked and PEGylated rMETase.

FIG. 4 is a graph depicting plasma enzyme activity following intravenous injection of naked and PEGylated rMETase in mice.

FIG. 5 is a graph depicting plasma methionine depletion following intravenous injection of naked and PEGylated rMETase in mice.

FIG. 6 is a graph depicting plasma PLP concentration with and without PLP pump implantation in mice.

FIG. 7 is a graph depicting plasma methionine depletion following PLP pump implantation and intravenous injection of naked and PEGylated rMETase.

FIG. 8 shows histographs depicting ratios of the plasma methionine level depleted by indicated PEGylated rMETase with and without PLP supplementation. See FIGS. 5 and 7 for original data.

Other features and advantages of the invention will be apparent from the following detailed description and from the claims.

### MODES OF CARRYING OUT THE INVENTION

Conjugation of proteins with polyethylene glycol (PEG) has been shown to increased serum half-life and reduced antigenicity of various therapeutic proteins (Kozlowski, A., and Harris, J. M., J. Control Release (2001) 72:217-224). PEG-conjugated proteins have shown enhanced solubility, decreased antigenicity, decreased susceptibility to proteolysis, and reduced rates of kidney clearance. PEG-conjugation has also been shown to enhance selective tumor targeting.

The FDA has approved the PEGylated forms of the protein therapeutics adenosine deaminase, asparaginase, α-interferon (IFN) and a growth hormone antagonist (Olson, K., et al. (1997) Poly(ethyleneglycol): Chemistry and Biological Applications (J. M. Harris, S. Zalipsky, eds.), ACS Books, Washington, D.C., pp. 170-181). PEG-α-IFN for treatment of hepatitis C (Park, C. W. G., and Chuo, M., (1999) U.S. Pat. No. 5,951,974) has recently been approved in two forms. Patients with refractory or recurrent acute lymphoblastic leukemia (ALL) are treated with a combination of PEG-asparaginase and methotrexate, vincristine, and prednisone (Aguayo, A, et al., Cancer (1999) 86:1203-1209). A genetic defect of enzyme adenosine deaminase (ADA) creates a deficiency that inhibits the development of the immune system, making patients vulnerable to almost any type of infection. PEG-ADA strengthened the immune system considerably in these patients (Pool, R., Science (1990) 248:305; and Hershfield, M. S., Clin. Immunol. Immunopathol. (1995) 76:S228-S232).

In addition to polyethylene glycol, any polyalkylene glycol can be used with the described invention. The term poly(alkylene glycol) refers to a polymer of the formula HO-[(alkyl)O]_{y}—OH, wherein alkyl refers to a C₁ to C₄ straight or branched chain alkyl moiety, including but not limited to methyl, ethyl, propyl, isopropyl, butyl, and isobutyl. Y is an integer greater than 4, and typically between 8 and 500, and more preferably between 40 and 500.

Any PLP-dependent enzyme can be used in the described methods. Examples of enzymes that can be used include: cystathionine γ-lyase; cystathionine γ-synthase; O-acetylhomoserine O-acetylserine sulfhydrylase; aspartate aminotransferase; thermophilic alanine racemase; psychrophilic alanine racemase; L-Methionine gamma-lyase (MGL); L-cystathionine beta-lyase; L-cystathionine gamma-synthase; D-amino acid transaminase; D-amino acid aminotransferase (D-AAT); leucine dehydrogenase; amino acid racemase; omega-amino acid transaminase; tryptophan synthase beta subunit-like PLP-dependent enzymes; nickel-iron hydrogenase; O-acetylserine sulfhydrylase; cystathionine beta synthase; dopa decarboxylase; 1-aminocyclopropane-1-carboxylate (ACC); L-threonine dehydratases; L-serine dehydratases; and methioninases.

Recombinant forms of the PLP-dependent enzyme may also be used with the disclosed methods. The activity of the PLP-dependent enzyme can be altered by mutating the amino acid sequence of the enzyme, or by replacing, or inactivating portions of the enzyme, using techniques known to one skilled in the art. PLP-dependent enzymes are described in: Yoshimura, T., et al., Biosci. Biotechnol. Biochem. (1996) 2:181-187; Motoshima, H., et al., J. Biochem. (Tokyo) (2000) 3:349-354; Grabowski, R., et al., Trends Biochem Sci. (1993) 8:297-300; and van Ophem, P. W., et al., Biochemistry (1999) 4:1323-1331, all of which are incorporated herein by reference.

In a preferred embodiment, a recombinant methioninase is used with the methods described herein. As with many other bacterial polypeptides and proteins, rMETase may be immunogenic in higher animals, which may limit the utility of rMETase, especially with regard to multiple dosing. Anti-METase antibodies may accelerate rMETase clearance and consequently reduce its therapeutic effectiveness. These antibodies may also reduce the enzyme potency by binding at or near the active site of the enzyme. Adverse allergic reactions to rMETase may also occur. The present data clearly confirms a decreased immunogenicity of the PEGylated forms of the enzyme, which may relate to the masking of the protein antigenic sites by the polymer modification. Regardless of the mechanism, modification of the enzyme greatly enhances the potential of therapeutic efficacy in the clinical setting.

To couple PEG to a protein, it is first necessary to activate the polymer. This is done by converting the hydroxyl terminus of the polymer to a functional group capable of reacting typically with lysine and N-terminal amino groups (Kozlowski, A., supra (2001)).

Each ethylene oxide unit of the PEG molecule associates with two to three water molecules. This interaction with water causes proteins decorated with PEG to behave as if it were five to ten times as large as a protein of comparable molecular weight (Kozlowski, A., supra (2001)). The clearance rate of PEGylated proteins is inversely proportional to molecular weight (Id.) In vivo, typically molecules with a molecular weight of about 20,000 or less are cleared relatively rapidly in the urine. Higher-molecular-weight PEGylated proteins, however are cleared more slowly in the urine and the feces (Yamaoka, T., et al., J. Pharm. Sci. (1994) 83:601-606).

PEGylation of rMETase is performed by activating PEG derivatives and reacting those derivatives with the target rMETase. The enzyme can be commercially and should be of pharmaceutical grade. A preferred enzyme is L-methionine α-deamino-γ-mercaptomethane lyase (methioninase, METase) [EC 4.4.1.11], which is found in is found in Pseudomonas, Aeromonas, Clostridium, Trichomonas, Nippostrongylus, Trichomonas vaginalis, Nippostrongylus brasiliensis, and Fusobacterium sp., but not in yeast, plants, or mammals. Recombinant and non-recombinant forms of METase can be used with the disclosed methods.

Activated PEG derivatives are combined with the METase to produce the modified enzyme. The PEG derivatives are preferably used in molar excess to the METase, although the molar ration of PEG derivatives to enzyme may be less than 1:1. Preferably, however, PEG derivatives to free lysines present in the enzyme amino acid sequence ratios are from about 1:1 to 1000:1. Specific examples of particular molar ratios include 1:1, 2:1, 3:1, 4:1, 5:1, 6:1, 7:1, 8:1, 9:1, 10:1, 15:1, 20:1, 25:1, 50:1, 75:1, 100:1, 110:1, 120:1, 130:1, 140:1, 150:1, 200:1, 500:1, 750:1, and 1000:1. Higher molar ratios of PEG derivative to enzyme are also contemplated.

Reaction conditions used to modify a particular METase preparation may vary depending of the source of the enzyme and the particular PEG derivative used to modify the enzyme. In a preferred embodiment, a given amount of the activated PEG is added to a METase solution in stepwise additions, typically at 30 minutes intervals. The PEGylation reactions are performed at a temperature that facilitates the modification of the enzyme without damaging the protein. In preferred embodiment, the modification reaction is carried out at 20-25° C. under gentle stirring for 90 minutes.

Unreacted activated PEG derivatives are typically removed from the solution using column chromatography. For example, in a preferred embodiment, modified protein is separated from unreacted derivatives using gel filtration column chromatography. Other types of chromatography, such as affinity chromatography and ion exchange chromatography can also be used. Following modification, the PEGylated enzyme preparation is purified to pharmaceutical standards and prepared in a manner suitable for therapeutic administration.

Pharmaceutical Preparations and Methods of Administration

The purified PEGylated METase is suitable for incorporation into pharmaceuticals that treat organisms in need thereof. The purified PEGylated METase is be processed in accordance with conventional methods of galenic pharmacy to produce medicinal agents for administration to subject mammals including humans. The purified PEGylated METase can be incorporated into a pharmaceutical product with and without further modification. The manufacture of pharmaceuticals or therapeutic agents that deliver the pharmacologically active compounds of this invention by several routes are aspects of the invention. For example, and not by way of limitation, DNA, RNA, and viral vectors having sequence encoding a METase of interest can be used to prepare the METase (i.e., rMETase) for use with the disclosed methods. Pharmaceutical compositions comprising the PEGylated METase can be administered alone or in combination with other active ingredients, such a various chemotherapeutic agents known to have efficacy in treating various neoplastic diseases.

The compounds of this invention can be employed in admixture with conventional excipients, such as pharmaceutically acceptable organic or inorganic carrier substances suitable for parenteral, enteral (e.g., oral) or topical application that preferably do not deleteriously react with the pharmacologically active ingredients of this invention. Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc. Many more suitable vehicles are described in Remington's Pharmaceutical Sciences, 15th Edition, Mack Publishing Co., Easton, Pa., pages 1405-1412 and 1461-1487(1975) and The National Formulary XIV, 14th Edition, Washington, American Pharmaceutical Association (1975), herein incorporated by reference. The disclosed pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like that preferably do not deleteriously react with the active compounds of the invention.

The effective dose and method of administration of a particular pharmaceutical formulation comprising a PEGylated METase can vary based on the individual needs of the patient and the treatment or preventative measure sought. Therapeutic efficacy and toxicity of such compounds can be determined by standard pharmaceutical procedures in cell cultures or experimental animals, e.g., ED₅₀ (the dose therapeutically effective in 50% of the population). For example, the PEGylated METase preparations disclosed herein can be tested in a mouse xenographic model. The effect of the modified enzyme on plasma methionine levels, tumor size, and tumor sensitivity to other chemotherapeutic agents can be determined in such a model system. The data obtained from these assays is then used in formulating a range of dosage for use with other organisms, including humans. The dosage of such compounds lies preferably within a range of circulating concentrations that include the ED₅₀ with no toxicity. The dosage varies within this range depending upon a number of variables including, for example the type of METase used, the type of neoplasm being treated, and the route of administration.

Normal dosage amounts of the PEGylated METase can vary from approximately 1 to 100,000 Units. In some embodiments, the dose of the PEGylated METase preferably produces reduction in serum methionine levels from approximately 40 μM to less than 10 μM. Desirable doses produce a blood concentration of methionine to about 1 to 10 μM.

The exact dosage is chosen by the individual physician in view of the patient to be treated. Dosage and administration are adjusted to provide sufficient levels of the PEG-modified METase enzyme to maintain the desired effect of reduced tumor methionine availability. Additional factors that can be taken into account include the severity of the disease, age and weight of the subject; diet, time and frequency of administration, drug combination(s), reaction sensitivities, and tolerance/response to therapy. Short acting pharmaceutical compositions are administered daily whereas long acting pharmaceutical compositions are administered every 2, 3 to 4 days, every week, or once every two weeks. Depending on half-life and clearance rate of the particular formulation, the pharmaceutical compositions of the invention are administered once, twice, three, four, five, six, seven, eight, nine, ten or more times per day.

Routes of administration of the pharmaceuticals of the invention include, but are not limited to, topical, transdermal, parenteral, gastrointestinal, transbronchial, and transalveolar. Transdermal administration is accomplished by application of a cream, rinse, gel, etc., capable of allowing the pharmacologically active compounds to penetrate the skin. Parenteral routes of administration include, but are not limited to, electrical or direct injection such as direct injection into a central venous line, intravenous, intramuscular, intraperitoneal, intradermal, or subcutaneous injection. Gastrointestinal routes of administration include, but are not limited to, ingestion and rectal. Transbronchial and transalveolar routes of administration include, but are not limited to, inhalation, either via the mouth or intranasally.

Pharmacological compositions comprising the PEGylated METase described herein are suitable for transdermal or topical administration as pharmaceutically acceptable suspensions, oils, creams, and ointments applied directly to the skin or incorporated into a protective carrier such as a transdermal device (“transdermal patch”). Examples of suitable creams, ointments, etc., can be found, for instance, in the Physician's Desk Reference. Examples of suitable transdermal devices are described, for instance, in U.S. Pat. No. 4,818,540 issued Apr. 4, 1989, to Chinen, et al., herein incorporated by reference.

Pharmacological compositions comprising the PEGylated METase described herein that are suitable for parenteral administration include, but are not limited to, pharmaceutically acceptable sterile isotonic solutions. Such solutions include, but are not limited to, saline and phosphate buffered saline for injection into a central venous line, intravenous, intramuscular, intraperitoneal, intradermal, or subcutaneous injection.

Pharmacological compositions comprising the PEGylated METase described herein that are suitable for transbronchial and transalveolar administration include, but not limited to, various types of aerosols for inhalation. Devices suitable for transbronchial and transalveolar administration of these are also embodiments. Such devices include, but are not limited to, atomizers and vaporizers. Many forms of currently available atomizers and vaporizers can be readily adapted to deliver compositions having the pharmacologically active compounds of the invention.

Pharmacological compositions comprising the PEGylated METase described herein that are suitable for gastrointestinal administration include, but not limited to, pharmaceutically acceptable powders, pills or liquids for ingestion and suppositories for rectal administration. Due to the ease of use, gastrointestinal administration, particularly oral, is a particularly preferred embodiment. Once the pharmaceutical comprising the PEGylated METase described herein has been obtained, it can be administered to a subject in need thereof.

The pharmaceutical compositions described herein comprise a PEGylated enzyme in a pharmaceutically acceptable carrier. Optionally, the pharmaceutical compositions may comprise additional compounds. For example, a variety of chemotherapeutic agents can be included in the pharmaceutical compositions described herein. Examples of suitable chemotherapeutic agents include carboplatin, cisplatin, cyclophosphamide, doxorubicin, daunorubicin, epirubicin, mitomycin C, mitoxantrone, 5-fluorouracil (5-FU), gemcitabine, methotrexate, camptothecin, irinotecan, topotecan, bleomycin, docetaxel, etoposide, paclitaxel, vinblastine, vincristine, vindesine, vinorelbine, genistein, trastuzumab, ZD1839; cytotoxic agents; apoptosis-inducing agents, cell cycle control inhibitors, verapamil, and cyclosporin A.

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed.

### EXAMPLES

### Preparation and Purification of PEGylated rMETase

Recombinant METase (rMETase) was provided by Shionogi Co., Ltd. (Osaka, Japan, Lot No. 8Y003). The production protocols of rMETase were as described previously (Tan, Y., supra (1997), Yoshioka, T., et al., Cancer Research (1998) 58:2583-2587). rMETase was formulated in 50 mM sodium phosphate buffer, pH 7.2, containing 10 μM PLP, with a protein concentration of 31 mg/ml and specific activity 50.7 U/mg. The rMETase was more than 95% pure by HPLC with tetramer/oligomer ratio 96.7/3.3 and endotoxin 0.06 EU/mg.

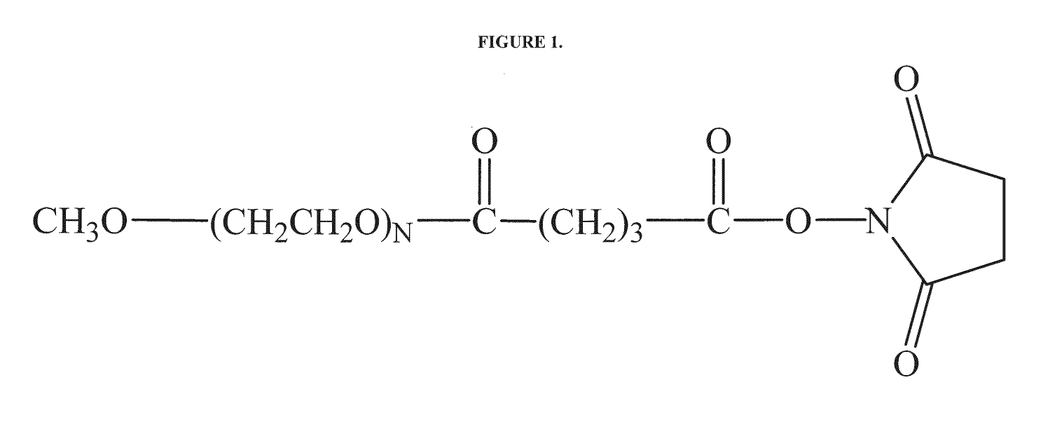

Methoxypolyethylene glycol succinimidyl glutarate-5000 (MEGC-50HS-PEG or MEGC-PEG) (NOF Corporation, Kawasaki-shi, Kanagawa, Japan, Lot No. M21514) had a polydispersity of 1.02, substitution 94.2%, dimer content 0.84% and purity by ¹H-NMR of 98.4%. The average molecular weight was 5461 Da. The chemical structure of MEGC-PEG is shown in FIG. 1.

DEAE Sepharose FF was purchased from AMERSHAM PHARMACIA BIOTECH (Piscataway, N.J., USA). Pre-cast, 10% tris-glycine gels were from NOVEX (San Diego, Calif., USA). Mini-osmotic pumps with a reservoir volume of 200 μl, pumping rate of 1.0 μl/hr and duration of 7 days (Model 2001) were purchased from DURECT CORPORATION (Cupertino, Calif., USA). PLP and other chemicals were purchased from SIGMA (St. Louis, Mo., USA).

The activated PEG derivative was used at a molar excess (1-5 fold) of PEG to free lysines in rMETase (32 per rMETase molecule), which corresponds to molar ratios of PEG to rMETase of 30-120/1. For each reaction, 120 mg/ml rMETase in 100 mM borate buffer (pH 8.8) was used. Based on 30-120/1 molar ratios of activated PEG versus rMETase (equal to 0.87-3.5/1 weight ratio of activated PEG versus rMETase), a given amount of the activated PEG was added to the rMETase solution with three stepwise additions at 30 minutes intervals. The PEGylation reactions were carried out at 20-25° C. under gentle stirring for 90 minutes.

In order to eliminate an excess of unreacted activated PEG, the resulting PEG-rMETase conjugate was applied on a Sephacryl S-300 HR gel filtration column (HIPREP 26/60, AMERSHAM PHARMACIA BIOTECH, Piscataway, N.J., USA) immediately after the PEGylation reaction. PEG-rMETase was eluted with 80 mM sodium chloride in 10 mM sodium phosphate, pH 7.4, containing 10 μM PLP at a flow rate of 120 ml/h.

The fractions containing the PEG-rMETase conjugate were further purified by DEAE Sepharose FF column (XK 16/15, AMERSHAM PHARMACIA BIOTECH, Piscataway, N.J., USA) to remove trace amounts of un-PEGylated rMETase. The column was equilibrated and eluted with 80 mM sodium chloride in 10 mM sodium phosphate pH 7.2, containing 10 μM PLP at a flow rate of 180 ml/h. The fraction containing the PEG-rMETase conjugate passed through the column and was collected. The final purified PEG-rMETase solution was concentrated by an AMICON CENTRIPREP® YM-30 (MILLIPORE CORP., Bedford, Mass., USA) and sterilized by filtration with a 0.22 μM membrane filter (FISHER SCIENTIFIC, Tustin, Calif., USA) and stored at −80° C.

Determination of Protein Content

Protein was measured with the Wako Protein Assay Kit (Wako Pure Chemical, Osaka, Japan) according to the instruction manual with slight modification (Watanabe, N., et al., Clin. Chem. (1986) 32:1551-1555). 50 μl of each sample or standard protein (BSA) was added to 3 ml of color-producing solution (pyrogallol red-molybdate complex) and vortexed well. The mixture was incubated at room temperature for 20 minutes without shaking and then measured for absorbance at 600 nm. The protein content of the sample was determined from the BSA standard calibration curve.

rMETase Activity Assay

rMETase activity was determined from α-ketobutyrate produced from L-methionine according to the method of Tanaka, H., et al., Biochemistry (1977) 16:100-106, with slight modification. 0.5 ml of sample diluted in 100 mM potassium phosphate buffer pH 8.0, containing 0.01% DTT, 1 mM EDTA Naₑ, 10 μM PLP and 0.05% Tween 80, was mixed with 0.5 ml of substrate solution containing 100 mM potassium phosphate buffer, pH 8.0, containing 25 mM L-methionine and 10 μM PLP in a glass test tube. The reaction mixture was vortexed immediately and incubated at 37° C. without shaking for precisely 10 minutes. The reaction was stopped by adding 0.5 ml of 50% TCA. The suspension was centrifuged at 13,000 rpm for 2 minutes. The supernatant (0.5 ml) was collected in a glass tube containing 1 ml 1 M acetate buffer, pH 5.0. Then, 0.4 ml MBTH solution containing 0.1% 3-methyl-2-benzothiazolinonehydrazone dihydrochloride monohydrate (Wako Pure Chemical, Osaka, Japan) was added to the tube, mixed well and incubated at 50° C. for 30 minutes. The absorbance of the reaction mixture was measured at 320 nm. The assay was carried out in triplicate. ΔE was calculated by subtracting the average absorbance of blanks from the average absorbance of the reaction mixture. The enzyme activity was calculated by the following equation: Activity (U/ml)=0.548 (1.07+2.2ΔE) ΔE. One unit of enzyme is defined as the amount of enzyme which produced 1 μM of α-ketobutyrate per minute at an infinite concentration of L-methionine.

SDS-Electrophoresis Analysis

SDS-PAGE analysis of PEG-rMETase was carried out using 10% NOVEX polyacrylamide-precasted tris-glycine gels in NOVEX tris-glycine buffer with SDS according to the instruction manual. Gels were stained with Coomassie brilliant blue.

Determination of PEGylation Degree of rMETase

The degree of modification of PEGylated rMETase was estimated both by the fluorescamine assay (Karr, L. J., et al., Methods Enzymol. (1994) 228:377-390) and by MALDI. For the fluorescamine assay, various amounts of rMETase and PEGylated rMETase in 2 ml of 0.1 M sodium phosphate buffer, pH 8.0 were mixed with 1 ml fluorescamine solution (0.3 mg/ml in acetone) and incubated for 5 min at room temperature. Samples were then assayed with a fluorescence spectrometer at 390 nm excitation and 475 nm emission. Results were plotted as fluorescence units versus concentration, with the slope of the line being determined by linear regression. The percent of PEGylated primary amines was determined according to the following formula:

1−(slope PEGylated rMETase/slope naked rMETase)×100.

MALDI analysis of naked and PEG-rMETase was performed at the Scripps Research Institute using a PerSeptive Biosystems VOYAGER-ELITE mass spectrometer.

Plasma Methionine Determination

The methionine level in the plasma was measured by pre-column derivitization, followed by HPLC separation (Jones, B. N., and Gilligan, J. P., J. Chromatogr. (1983) 266:471-482). Briefly, 10 μl of plasma sample or methionine standard was precipitated with 30 μl of acetonitrile, followed by centrifugation at 10,000 rpm for 5 minutes. 10 μl of the supernatant was mixed with 5 μl of a fluoraldehyde derivative reagent, o-phthaldialdehyde, for 1 minute at room temperature, followed by addition of 150 μl of 0.1 M sodium acetate, pH 7.0. 20 μl of the reaction mixture was loaded on a reversed-phase Supelcosil™ LC-18DB column (25 cm×4.8 cm, particle size 5 μm (Supelco, Bellefonte, Pa., USA). The amino acid derivatives were separated by using a gradient elution of 40-60% solution B (Methanol) in solution A (tetrahydrofuran/methanol/0.1M sodium acetate. pH 7.2; 5/95/900) at a flow rate of 1.5 ml/minute. A fluorescence spectrophotometer was used for detection: excitation at 350 nm and emission at 450 nm. The plasma methionine was identified by the retention time of a methionine standard solution and quantitated according to a methionine standard curve.

Determination of Plasma Pyridoxal 5′-Phosphate

PLP in plasma was determined by HPLC using derivitization with sodium bisulfite in the mobile phase (Deitrick, C. L., et al., J. Chromatogr B Biomed Sci Appl. (2001) 751:383-387). Briefly, the plasma sample and PLP standard solutions were mixed with an equal volume of 0.8 M HCIO₄ as deproteinizing agent, and vortexed vigorously. After centrifuging at 15000 rpm for 5 min at 4° C., the supernatants were taken and transferred to new vials. 50 μl of the supernatant was loaded on a reversed phase Cosmosil™ 5C18-AR-II column (4.6×150, Nacalai Tesque, Japan). The column was eluted with a gradient elution of 20-80% mobile phase B (30% acetonitrile/water v/v) in mobile phase A (0.1 M potassium dihydrogen phosphate buffer containing 0.1 M sodium perchlorate and 0.5 g/L sodium bisulfite, pH 3.0) at a flow rate of 1.0 ml/minute. A fluorescence spectrophotometer was used for detection: excitation at 300 nm and emission at 400 nm. The PLP peak was identified by the retention time of a PLP standard. The concentration of plasma PLP was calculated using a calibration curve.

Pharmacokinetics and Methionine Depletion Efficacy In Vivo

Athymic nude (nu/nu) mice aged 4 weeks (20-25 g) were used for the study with 4 mice per group. Osmotic mini-pumps filled with 250 μl PLP (0.5 g/ml) were implanted subcutaneously. Twenty-four hours after pump implantation, 80 units of native rMETase or PEG-rMETase in 0.5 ml PBS, pH 7.4, were injected via the tail vein in mice with or without PLP pumps. 400 μl of blood was collected from the retroorbital plexus of each animal using heparinized capillary tubes. Blood was collected prior to injection, and 1, 2, 4, 8, 24, 48, 72, 96 and 120 hours post injection. The plasma was separated and stored in small aliquots at −80° C. The plasma enzyme activity, methionine concentration and PLP level in the plasma, collected at the different time points, were measured as described above.

Determination of Plasma Anti-rMETase Antibody

BALB/c male mice were grouped randomly at 5 per group. Each mouse received three i.p. injections of 0.2 ml (200 μg) naked or PEG-rMETase emulsified in Freund's complete adjuvant (FCA) at weekly intervals. Two weeks following the last injection, a booster injection of the rMETase or PEG-rMETase was given to each mouse. Blood samples were collected two weeks after the booster injection, and plasma was separated and stored at −80° C.

Plasma anti-rMETase antibody was measured using a sandwich ELISA technique. 100 μl 200 μg/ml rMETase in 0.1 M carbonate coating buffer, pH 9.5, was added to each well of a 96-well microplate and incubated at 4° C. overnight. The plate was washed three times with PBS washing buffer, pH 7.4, containing 0.05% Tween-20, and blocked for 2 hours at room temperature with 200 μl of PBS assay buffer, pH 7.4, containing 10% FBS. After washing three times, 100 μl of 10-fold serial dilutions of the plasma samples in PBS assay buffer were added to appropriate wells and incubated for 2 hours at room temperature, followed by washing. 100 μl optimally diluted goat anti-mouse IgG and IgM subtypes conjugated with horseradish peroxidase (SIGMA, St. Louis, Mo., USA) were added to each well. The plate was incubated for 1 hour at room temperature and washed three times. 100 μl substrate solution containing O-phenylenediamine dihydrochloride (OPD) and hydrogen peroxide (SIGMA, St. Louis, Mo., USA) were added to each well, followed by 30 min incubation at room temperature. 50 μl 2N sulfuric acid were added to each well to stop the color reaction. The absorbance of each well was measured at 492 nm. The antibody titer was determined as the highest plasma dilution at which the extinction at 492 nm generated in the well of the immune plasma exceeded twice the extinction generated in those wells with negative control plasma.

SDS-PAGE Analysis of PEGylated rMETase

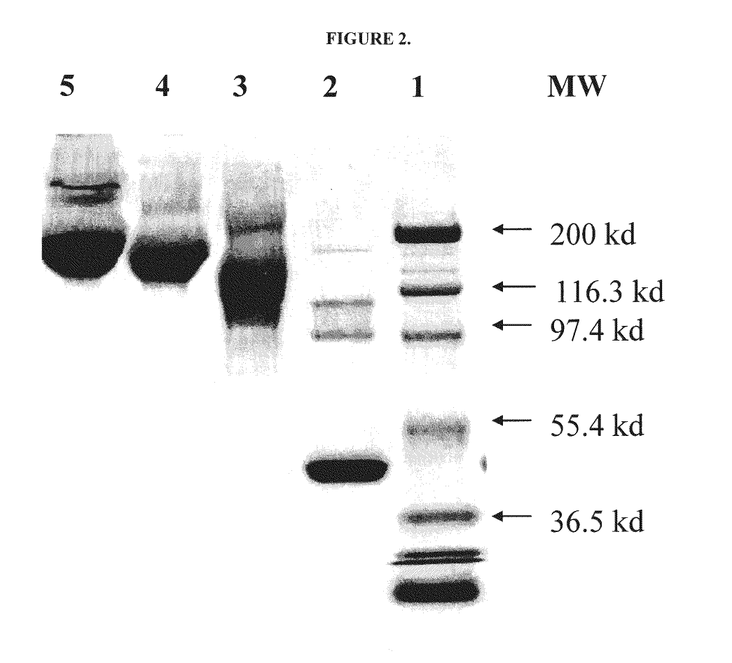

Three rMETase conjugates were prepared using molar ratios of PEG/rMETase of 30/1, 60/1 and 120/1. The extent of PEG conjugation to rMETase and the purity of PEGylated rMETase conjugates were determined by SDS-PAGE (FIG. 2). All rMETase subunits were PEGylated after 90 minutes reaction at room temperature when the above molar ratios were used, since no non-PEGylated rMETase subunits were detected by SDS-PAGE (FIG. 2). The purified PEGylated rMETases were run on a 10% SDS-PAGE gel. More PEG chains were conjugated to rMETase with increasing molar ratios of PEG to rMETase as seen by SDS-PAGE (FIG. 2). The gel was stained with Coomassie brilliant blue, numbers on the right indicate molecular mass. Lane 1: Molecular weight markers. Lane 2: Naked rMETase. Lane 3: PEG/rMETase 30. Lane 4: PEG/rMETase 60. Lane 5: PEG/rMETase 120. Broad bands were observed on the gels, indicating the heterogeneity of PEGylated rMETase conjugates at low PEG/rMETase ratios. When higher molar PEG/rMETase ratios were used in the reaction, less heterogeneity of PEGylated rMETase conjugates was observed.

Determination of PEGylation Degree by Fluorescamine Assay and MALDI

The PEGylation degree of three PEGylated rMETase conjugates are shown in Table 1.

TABLE 1
Determination of PEGylation degree of PEGylated rMETase conjugates

| | | MALDI | | |
|---|---|---|---|---|
| | | | | PEG's per |
| | Fluorescamine | Molecular | | rMETase |
| | Assay (%) | mass | | monomer |
| Naked rMETase | 0 | 42.3 | kD | 0 |
| PEG/rMETase 30 | 33 | 48.3-75.3 | kD | 1-6 |
| PEG/rMETase 60 | 61 | 59.8-81.0 | kD | 3-7 |
| PEG/rMETase 120 | 81 | 82.0-91.9 | kD | 7-9 |

The degree of PEGylation of naked and PEGylated rMETase was determined using the fluorescamine assay and MALDI as described above. The results of fluorescamine assay were expressed as the percentage of PEGylated lysine groups in rMETase. The MALDI results were expressed as both the total molecular mass of PEGylated rMETase monomer and the calculated number of conjugated PEG polymers per rMETase monomer. Each PEG polymer attached to rMETase contributes approximately 5 kD to the total molecular mass of PEGylated rMETase monomer.

The fluorescamine assay indicated approximately 33%, 61% and 81% of free lysines in rMETase were coupled with PEG chains at ratios of PEG/rMETase 30, PEG/rMETase 60 and PEG/rMETase 120, respectively. The data indicated corresponds to an average of 3, 5 and 7 PEGylated lysines in each rMETase subunit, respectively, using the above coupling ratios.

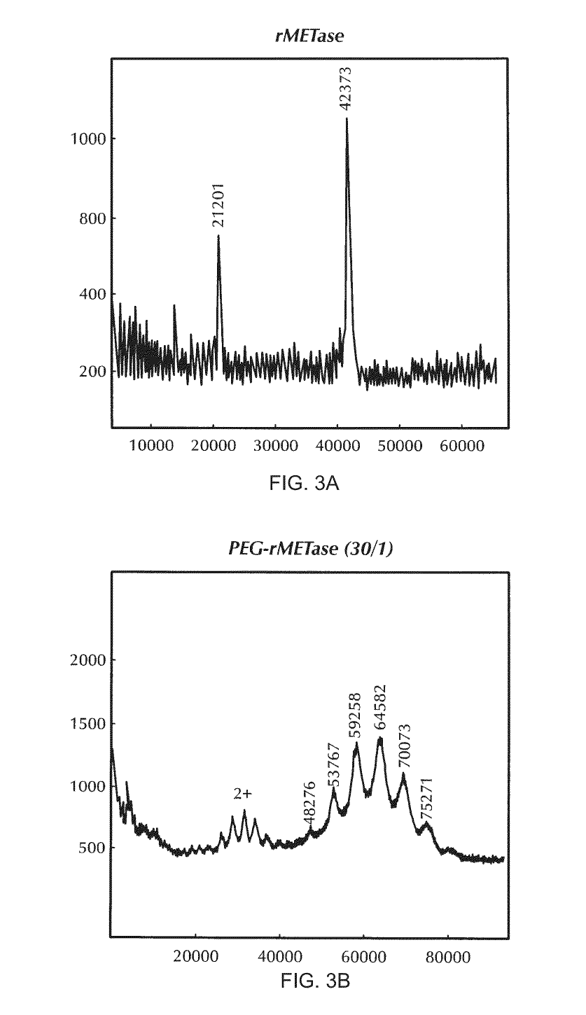

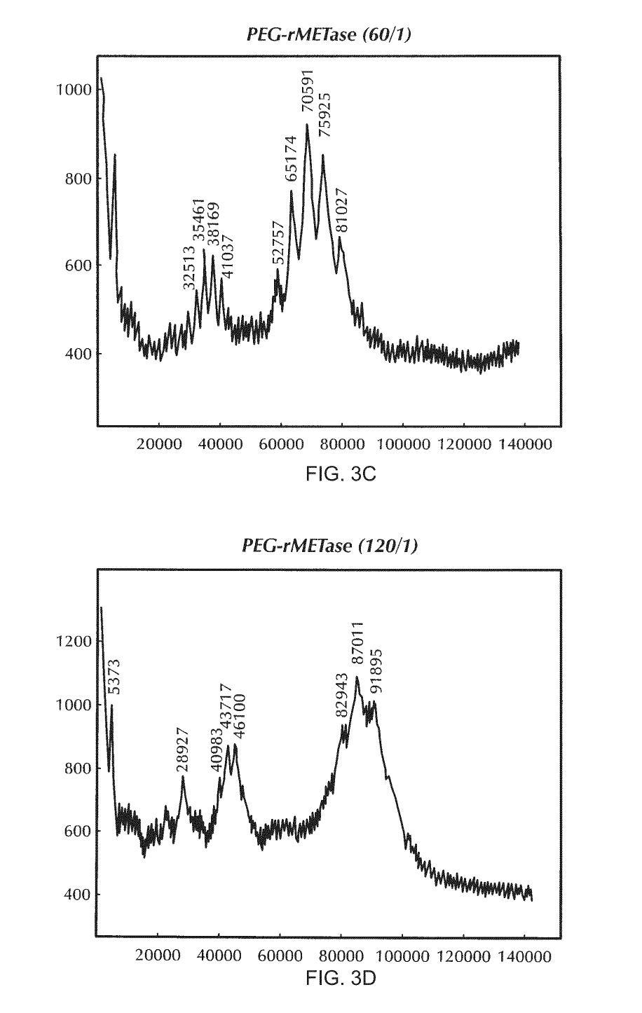

MALDI analysis also demonstrated that a series of signal peaks were observed at an average of molecular mass of 64582, 70591 and 87011 Da for the three coupling ratios used, respectively (FIG. 3). MALDI analysis was performed using a PerSeptive Biosystems Voyager-Elite mass spectrometer. The last serial peaks at 48276-75271, 59757-81027 and 82043-91895 Da represent naked rMETase, PEG-rMETase (30/1), PEG-rMETase (60/1) and PEG-rMETase (120/1) ion signals, respectively. The series of peaks before the naked rMETase and PEG-rMETase peaks are doubly charged species derived from the above parent ions. These data are consistent with the covalent attachment of an average of 4, 6 and 8 PEG units to each subunit of rMETase respective to the three ratios used. The serial peaks of PEGylated rMETase in the MALDI spectrum reflect the distribution heterogeneity arising from PEGylation of rMETase. It was found that at a higher PEGylation degree, fewer signal peaks in MALDI were observed, indicating less heterogeneity. For example, the PEG/rMETase 120 conjugate was the least heterogeneous (FIG. 3). Both MALDI and the fluorescamine assay demonstrate that an increase in PEG/rMETase molar ratios in the PEGylation reaction resulted in an increase in PEGylation of rMETase subunits.

Plasma Circulating Half-Life of PEGylated rMETase

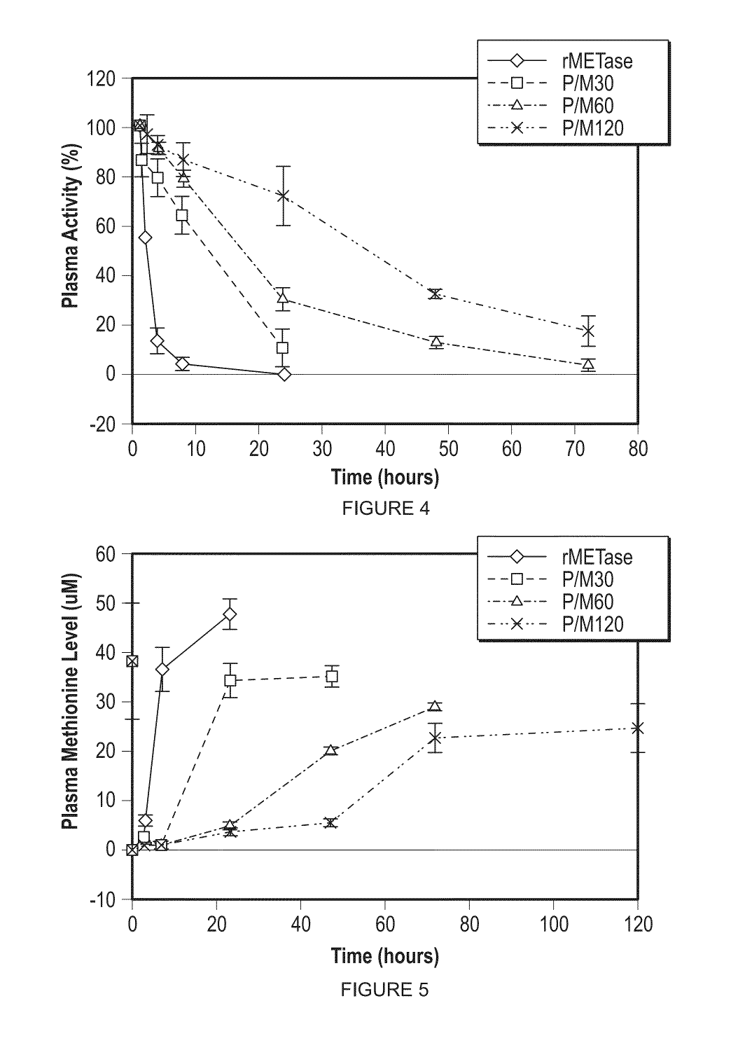

Plasma enzyme activity of naked rMETase decreased rapidly and was undetectable in blood 24 hours after injection of 80 units per mouse. However, PEGylated rMETase demonstrated a significant pharmacokinetic improvement. Plasma enzyme activity was detectable until 72 hours, when PEG/rMETase 60 and PEG/rMETase 120 were evaluated. The half-life for naked rMETase was 2 hours in contrast to the half-life of 12 hours, 18 hours and 38 hours for PEG/rMETase 30, PEG/rMETase 60 and PEG/rMETase 120, respectively (FIG. 4, Table 2).

TABLE 2
Plasma half-life of naked and PEGylated rMETase in mice

| | Plasma half-life in mice |
|---|---|
| Naked rMETase | 2 hours |
| PEG/rMETase 30 | 12 hours |
| PEG/rMETase 60 | 18 hours |
| PEG/rMETase 120 | 38 hours |

FIG. 4 shows the plasma enzyme activity following intravenous injection of naked and PEGylated rMETase in mice. The mice were injected intravenously with 80 U naked or the indicated PEGylated rMETase. Blood samples were collected at different time points and measured for plasma enzyme activity as described above.

Plasma Methionine Depletion Efficacy of PEGylated rMETase

The plasma methionine was depleted from a baseline of 40 μM to less than 5 μM within 1 hour by 80 U naked and the three PEGylated conjugates (FIG. 5). However, PEG/rMETase 30, PEG/rMETase 60 and PEG/rMETase 120 depleted the plasma methionine level below 5 μM for 8 hours, 24 hours and 48 hours, respectively, which is 2-, 6- and 12-fold longer than naked rMETase (Table 3).

TABLE 3
Maximum period of plasma methionine depletion below 5 μM by naked
and PEGylated rMETase with and without PLP supplementation

Max. Period
of MET
depletion
below
5 μM (hours)

| | PLP⁻ | PLP⁺ |
|---|---|---|
| Naked rMETase | 4 | 4 |
| PEG/rMETase 30 | 8 | 48 |
| PEG/rMETase 60 | 24 | 48 |
| PEG/rMETase 120 | 48 | 72 |

80 U of naked or PEGylated rMETase were injected i.v. in each mouse with or without PLP supplementation. Blood samples were collected at different time points and measured for enzyme activity and methionine concentration as described in the Materials and Methods. Plasma half-life was calculated from the plasma enzyme concentration-time profiles (FIG. 4). Maximum period of methionine depletion below 5 μM is calculated from the plasma methionine levels in FIGS. 5 and 7.

FIG. 5 shows plasma methionine depletion following intravenous injection of naked and PEGylated rMETase in mice. The mice were injected i.v. with 80 U of naked or the indicated PEGylated rMETase. Blood samples were collected at different time points and measured for plasma methionine concentration as described above.

Effect of PEGylation on In Vivo PLP Dependence of rMETase

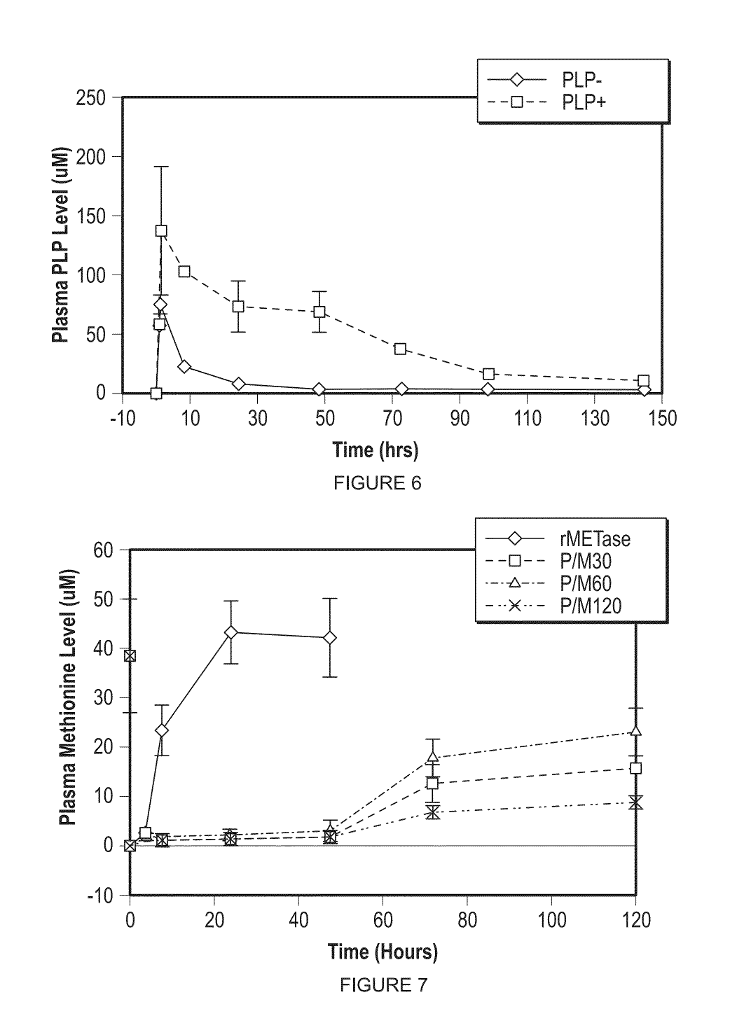

PLP mini-osmotic pump implantation significantly increased plasma PLP concentration (FIG. 6). In the experiments that produce the data of FIG. 6, mini-osmotic pumps filled with 250 μl of 0.5 g/ml PLP were implanted subcutaneously in mice before intravenous injection of naked or PEGylated rMETase. Blood samples were collected at different time points and measured for plasma PLP concentration as described in the Materials and Methods. PLP⁻ indicates the animals without PLP pumps. PLP⁺ indicates the animals with PLP pumps.

FIG. 7 shows plasma methionine depletion following PLP pump implantation and intravenous injection of naked and PEGylated rMETase. In these experiments, 80 U of naked or PEGylated rMETase was injected intravenously in animals with and without PLP supplementation from implanted mini-osmotic pumps. Blood samples were taken at different time points and measured for plasma methionine concentration. Plasma methionine depletion with PLP supplementation was prolonged significantly (FIG. 7) as compared to the methionine depletion without PLP supplementation (FIG. 5), indicating PLP dependence for the in vivo methionine-depletion efficacy of PEG-rMETase. The plasma methionine levels depleted by the three PEGylated rMETase conjugates were maintained below 2 μM for 48 hours and below 4 μM for 72 hours in case of PEG/rMETase 120 with PLP supplementation (Table 3). At 120 hours after injection of PEG/rMETase, the plasma methionine level depleted by PEG/rMETase 30, PEG/rMETase 60 and PEG/rMETase 120 still remained at 40%, 57% and 22% of normal baseline, respectively (Table 3).

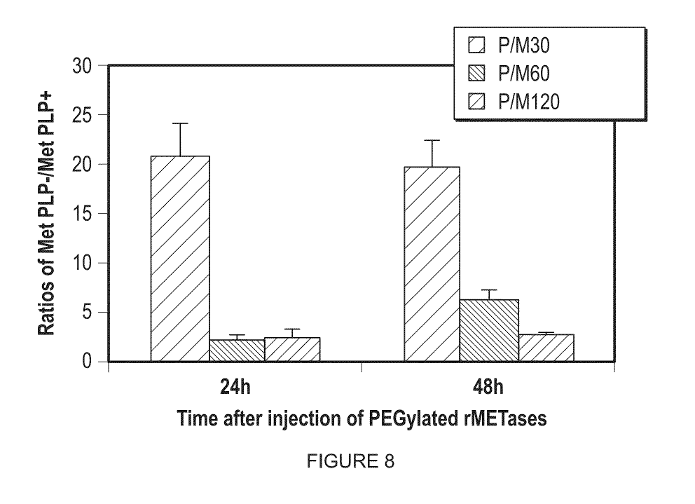

To quantitate the effect of PEGylation on PLP dependence of rMETase, the ratios of the plasma methionine concentration depleted by PEGylated rMETase without PLP supplementation (PLP) (FIG. 5) versus that depleted by PEGylated rMETase with PLP supplementation (PLP⁺) (FIG. 7) at various time points (FIG. 8) were calculated. FIG. 8 shows the ratios of the plasma methionine level depleted by indicated PEGylated rMETase with and without PLP supplementation. (See FIGS. 5 and 7 for original data.) Low ratios indicated similar methionine depletion under PLP⁻ and PLP⁺ conditions, indicating that PEGylation can decrease the PLP dependence of rMETase. These ratios showed that PEG/rMETase 60 and PEG/rMETase 120 had lesser PLP dependence than PEG/rMETase 30 (FIGS. 5, 7 and 8), suggesting a protective effect of PEGylation on retention of PLP by rMETase.

Effect of PEGylated rMETase on Plasma Anti-rMETase Specific Antibodies

The antigenicity of naked and PEGylated rMETase was evaluated by measuring plasma anti-rMETase-specific IgG and IgM antibodies in the mice immunized with naked or PEGylated rMETase in the presence of Freund's Complete Adjuvant (FCA). As shown in Table 4, the plasma obtained from the mice immunized with PEG/rMETase 30, PEG/rMETase 60 and PEG/rMETase 120 produced IgG antibody titers of 10⁻⁷, 10⁻⁶ and 10⁻⁴, respectively, as compared to the titer of 10⁻⁸ from naked rMETase. For IgM antibody, an antibody titer of 10⁻³ was detected in the mice immunized with three PEGylated rMETase conjugates, which was lower than the antibody titer of 10⁻⁴ from naked rMETase.

TABLE 4
Plasma anti-rMETase specific antibody titers induced
by naked or PEGylated rMETase in mice

| | IgG | IgM |
|---|---|---|
| Naked rMETase | 10⁻⁸ (5) | 10⁻⁴ (4), 10⁻⁵ (1) |
| PEG/rMETase 30 | 10⁻⁷ (4), 10⁻⁶ (1) | 10⁻³ (5) |
| PEG/rMETase 60 | 10⁻⁶ (5) | 10⁻³ (5) |
| PEG/rMETase 120 | 10⁻⁴ (4), 10⁻⁵ (1) | 10⁻³ (5) |

IgG and IgM anti-rMETase antibody titers of the plasma of the mice immunized with naked or indicated PEGylated rMETase in the presence of FCA were determined using ELISA as described in the Materials and Methods. The figures in parentheses represents the numbers of mice.

It was found that the higher the rMETase concentration in the PEGylation reaction, the greater the modification of rMETase. Therefore, the rMETase concentration was brought to 120 mg/ml rMETase.

Heterogeneity of PEGylated rMETase was observed by both SDS-PAGE and MALDI. PEG, being a synthetic polymer, is polydispersed, which contributes to the heterogeneity of PEGylated conjugates. Ideally, a polydispersivity value (Mw/Mn) ranging approximately from 1.01 for low molecular weight oligomers (3-5 kD), to 1.2 for high molecular weight (20 kD) may be expected for PEGylation of proteins and peptides (Veronese, F. M., Biomaterials (2001) 22:405-417). Besides polydispersivity of PEG, it was found that the PEG/rMETase molar ratio influenced the heterogeneity of the resulting conjugate (FIG. 2, FIG. 3, and Table 1) with higher PEG/rMETase molar ratios resulting in less heterogeneity of PEGylated rMETase.

Pharmacokinetic data of the PEGylated rMETase conjugates indicated that the plasma circulating time depended on the PEGylation degree (FIG. 4). Compared to the plasma half-life of 40 minutes for the PEGylated rMETase with M-SPA-PEG (Motoshima, H., et al., J. Biochem. (2000) 128:349-354), rMETase PEGylated with MEGC-PEG demonstrated a longer circulating time in blood. The half-life of PEG/rMETase 30, PEG/rMETase 60 and PEG/rMETase 120 was prolonged to 12 hours, 18 hours and 38 hours, respectively (Table 2). This improved pharmacokinetic property may reflect the higher PEGylation efficiency of MEGC-PEG than M-SPA-PEG for rMETase.

A plasma methionine concentration of 5 μM was used as an end point depletion level since it was reported that plasma methionine depletion below 5 μM was an effective therapeutic level of rMETase efficacy using mouse models of human cancer (Kokkinakis, D. M., Cancer Research (2001) 61:4017-4023). Without PLP supplementation this level of methionine depletion could be achieved for 8 hours by PEG/rMETase 30; 24 hours by PEG/rMETase 60; and 48 hours by PEG/rMETase 120. With supplementation, depletion to less than 5 μM methionine could be achieved for 48 hours for PEG/rMETase 30; for 48 hours by PEG/rMETase 60; and 72 hours by PEG/rMETase 120.

The protective effect of PEGylation on the apparent in vivo retention of PLP by rMETase was an unexpected result as was the rapid loss in vivo of PLP by naked rMETase and low-degree PEGylated rMETase. PLP in vitro is relatively tightly bound to rMETase (Han, Q., et al., Clinical Chemistry (2002) 48:1560-1564), but in vivo PLP appears to readily dissociate from rMETase. The recognition of the PLP effect which resulted in evidence of in vivo stabilization and retention of enzyme and prolonged efficacy in terms of reduction in plasma methionine levels is a potentially important therapeutic issue. The mechanism of dissociation of PLP from rMETase and apparent inhibition of dissociation by PEGylation will be investigated in future studies.

Plasma anti-rMETase-specific antibody determination showed that PEGylated rMETase could reduce the antigenicity of rMETase in mice. Plasma IgG antibody is a critical antibody subtype which is related to hypersensitivity reactions and antibody neutralization of foreign proteins in vivo. PEG/rMETase demonstrated a significant decrease in antigenicity. For example, plasma anti-rMETase IgG antibody titer was reduced to 10⁻⁴ by PEG/rMETase 120 as compared to 10⁻⁸ for naked rMETase. Reduction in plasma IgG antibody depended on the number of PEG-derivatized amino groups, indicating the decreased antigenicity of PEGylated rMETase is a consequence of masking the protein antigenic sites by the polymer modification. The present data confirms a decreased immunogenicity of the PEGylated forms of the enzyme, which may relate to the masking of the protein antigenic sites by the polymer modification. The modification should greatly enhance the potential of therapeutic efficacy in the clinical setting.

Elevated minimum methionine dependence of many types of tumor cells relative to normal cells has been demonstrated (Tan, Y, et al., supra (1997); Hoffman, R. M., and Erbe, R. W., Proc. Natl. Acad. Sci. USA (1976) 73:1523-1527; Hoffman, R. M., Biochem. Biophys. Acta (1984) 738:49-87; Mecham, J. O., et al., Biochem. Biophys. Res. Commun. (1983) 117:429-434; Guo, H. Y., et al., Cancer Res. (1993) 53:2479-2483; Kreis, W., and Goodenow, M., Cancer Res. (1978) 38:2259-2262′ Guo, H., et al., Cancer Res. (1993) 53:5676-5679; Goseki, N., et al., Jpn. J. Cancer Res. (1995) 86:484-489; Goseki, N., et al., Cancer (1992) 69:1865-1872; Lishko, V. K., et al., Protein Expression and Purification (1993) 4:529-533; Tan, Y., et al., Anticancer Res. (1996) 16:3931-3936; and Tan, Y., et al., Anticancer Res. (1996) 16:3937-3942). L-methionine α-deamino-γ-mercaptomethane lyase (methioninase, METase) [EC 4.4.1.11] from Pseudomonas putida has been previously cloned and produced in Escherichia coli (Tay, Y., et al., supra (1997); Inoue, H., et al., J. Biochem. (1995) 117:1120-1125; and Hori, H., et al., Cancer Res. (1996) 56:2116-2122) to target the methionine dependence of tumor cells. rMETase is found in Pseudomonas (Pp), Aeromonas, and Clostridium, but not in yeast, plants, or mammals (Motoshima, H., et al., supra (2000)). rMETase is a homotetrameric pyridoxal 5′-phosphate enzyme of 172 kDa molecular mass. The biochemical reaction catalyzed by rMETase is shown below:

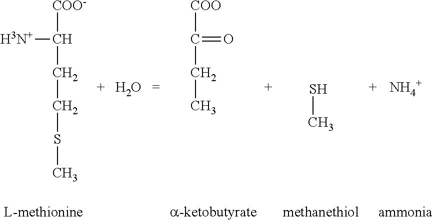

rMETase has 398 amino acid residues per subunit. The amino acid sequence of rMETase is homologous to the γ-family of PLP enzymes that catalyze α,γ-elimination and γ-replacement reactions, such as cystathionine γ-lyase, cystathionine γ-synthase, and O-acetylhomoserine O-acetylserine sulfhydrylase (Inoue, H., et al., Biosci. Biotechnol. Biochem. (2000) 64:2336-2343) in rMETase. Tyrosine 114 has been shown to be important in γ-elimination of the substrate (Inoue, H., et al., supra (2000)).

rMETase has been crystallized (Motoshima, H., et al., supra (2000), and Sridhar, V., et al., Acta Cryst. (2000) D56:1665-1667). The structure of rMETase has been determined at 1.7 Å resolution using synchrotron radiation diffraction data and found to be a homotetramer with 222 symmetry. Two monomers associate to build the active dimer. The spatial fold of the subunits, have three functionally distinct domains. Their quaternary arrangement, is similar to those of L-cystathionine β-lyase and L-cystathionine γ-synthase from E. coli (Motoshima, H, et al., supra (2000)).

Numerous modifications may be made to the foregoing systems without departing from the basic teachings thereof. Although the invention has been described in substantial detail with reference to one or more specific embodiments, those of skill in the art will recognize that changes may be made to the embodiments specifically disclosed in this application, yet these modifications and improvements are within the scope and spirit of the invention, as set forth in the claims which follow. All publications or patent documents cited in this specification are incorporated herein by reference as if each such publication or document was specifically and individually indicated to be incorporated herein by reference.

Citation of the above publications or documents is not intended as an admission that any of the foregoing is pertinent prior art, nor does it constitute any admission as to the contents or date of these publications or documents.

1. A method of increasing the serum half-life of a methioninase by coupling the methioninase to a polyalkene glycol, wherein at least 33% of free lysine residues in the methioninase are coupled with polyalkene glycol units.

2. The method of claim 1, wherein the half-life of recombinant methioninase is adjusted by altering the amount of polyalkene glycol that is coupled to the methioninase.

3. The method of claim 2, wherein the molar ratio of polyalkene glycol to methioninase is approximately 30:1.

4. The method of claim 2, wherein the molar ratio of polyalkene glycol to methioninase is approximately 60:1.

5. The method of claim 2, wherein the molar ratio of polyalkene glycol to methioninase is approximately 120:1.

6. The method of claim 1, wherein the methioninase is recombinantly produced.

7. The method of claim 1, wherein the methioninase is L-methionine α-deamino-γ-mercaptomethane lyase.

8. The method of claim 1, wherein the methioninase coupled with polyalkene glycol is administered with pyridoxal 5′-phosphate (PLP) to a subject.
